(19) United States
(12) Patent Application Publication
(10) Pub. No.: US 2014/0377386 A1
(43) Pub. Date: Dec. 25, 2014

(54) PLANT-BASED INHIBITORS OF KETOHEXOKINASE FOR THE SUPPORT OF WEIGHT MANAGEMENT

(71) Applicants: Jatinder Rana, Grand Rapids, MI (US); Russell Keith Randolph, Anaheim, CA (US); Jeffrey Scholten, Grand Rapids, MI (US); MyPhuong Thi Le, Denver, CO (US); Richard J. Johnson, Centennial, CO (US); Miguel Angel Lanaspa Garcia, Denver, CO (US)

(72) Inventors: Jatinder Rana, Grand Rapids, MI (US); Russell Keith Randolph, Anaheim, CA (US); Jeffrey Scholten, Grand Rapids, MI (US); MyPhuong Thi Le, Denver, CO (US); Richard J. Johnson, Centennial, CO (US); Miguel Angel Lanaspa Garcia, Denver, CO (US)

(73) Assignees: Access Business Group International LLC, Ada, MI (US); The Regents of the University of Colorado, Denver, CO (US)

(21) Appl. No.: 14/305,876

(22) Filed: Jun. 16, 2014

Related U.S. Application Data
(60) Provisional application No. 61/836,843, filed on Jun. 19, 2013.

(51) Int. Cl. A61K 36/73; A61K 36/38; A61K 36/185; A61K 36/487; A61K 36/539; A61K 36/12; A61K 36/19; A61K 36/62; A61K 36/75; A61K 36/23; A61K 36/605; A61K 36/232; A61K 36/44

(52) U.S. Cl. CPC A61K 36/73; A61K 36/232; A61K 36/38; A61K 36/185; A61K 36/487; A61K 36/539; A61K 36/44; A61K 36/19; A61K 36/62; A61K 36/75; A61K 36/23; A61K 36/605; A61K 36/12
USPC 424/745; 424/725; 424/769; 424/757; 424/765

(57) ABSTRACT

A composition for inhibiting ketohexokinase, for example, ketohexokinase-C (KHK-C) activity, may include a plant extract exhibiting at least IC50 (i.e., 50% KHK-C inhibition at a concentration in the range of from about 0.1 μg/mL to about 1000 μg/mL. The composition may be in a form suitable for oral ingestion. A method for inhibiting KHK-C activity in a subject may include administering a plant extract that exhibits at least 50% KHK-C inhibition at a concentration from about 0.1 μg/mL to about 1000 μg/mL. The administering may be done to treat or prevent at least one of sugar addiction, obesity, or metabolic syndrome. The administering may be done to provide a diminished craving in the subject from at least one member selected from the group consisting of craving of sugar, fructose, fructose-containing sugars, carbohydrates, and combinations thereof. The subject may be pre-diabetic, diabetic and or insulin resistant.

### RELATED APPLICATIONS

The present patent document claims the benefit of the filing date under 35 U.S.C. §119(e) of Provisional U.S. Patent Application Ser. No. 61/836,843, filed Jun. 19, 2013, which is hereby incorporated by reference.

### BACKGROUND

The present disclosure relates generally to inhibitors of ketohexokinase and, more particularly, to plant-based inhibitors of ketohexokinase and the use of such plant-based inhibitors for the support of weight management.

The intake of added sugars, especially sucrose and high fructose corn syrup (HFCS), has increased markedly over the last century in developed countries around the world. Epidemiological studies strongly associate the consumption of dietary sugar with the incidence of metabolic syndrome and obesity. Experimentally, the administration of fructose to rats has been shown to induce all features of metabolic syndrome, weight gain, and increased body fat.

Ketohexokinase (KHK) is an enzyme found in the liver, the renal cortex, and the small intestine that is involved in the metabolism of fructose in the body. KHK catalyzes the phosphorylation of fructose by adenosine triphosphate (ATP) to produce fructose-1-phosphate and adenosine diphosphate (ADP) according to the following reaction:

ATP+D-fructose→ADP+D-fructose-1-phosphate

Fructose-1-phosphate is then metabolized by aldolase B to generate various substrates. The phosphorylation of fructose consumes ATP and generates ADP.

Fructose is distinct from other sugars in that it causes transient intracellular ATP depletion in the liver prior to generating energy. This occurs with regularly ingested oral doses of fructose, even in humans. The mechanism may be due to the rapid phosphorylation of fructose by KHK. It is believed that such rapid phosphorylation of fructose is possible because KHK does not have a negative feedback system like hexokinases (e.g., glucokinase), which catalyze the phosphorylation of hexoses (e.g., glucose). KHK consumes ATP rapidly, resulting in activation of adenosine monophosphate (AMP) deaminase and the generation of uric acid, which increases in both hepatocytes and transiently in the circulation. ATP depletion by KHK is critical for fatty liver formation.

### SUMMARY

In one example, a composition for inhibiting ketohexokinase, for example, for inhibiting ketohexokinase-C (KHK-C) activity, may include a plant extract exhibiting at least IC50 (i.e., at least 50% KHK-C inhibition at a concentration from about 0.1 μg/mL to about 1000 μg/mL). The plant extract may be obtained from a plant from a genus selected from the group consisting of Angelica, Cratoxylum, Myrica, Psoralea, Scutellaria, Diospyros, Andrographis, Nymphaea, Chloroxylon, Petroselinum, Morus, Pteris, Garcinia, and Malus. The plant extract may be obtained from a plant selected from the group consisting of Angelica archangelica, Cratoxylum prunifolium, Myrica cerifera, Psoralea corylifolia, Scutellaria baicalensis, and Diospyros attenuata, Andrographis paniculata, Nymphaea lotus, Chloroxylon swietenia, Petroselinum crispum, Morus alba, Pteris wallichiana, Garcinia mangostana, and Malus domestica. The plant extract may include a compound selected from the group consisting of Osthol, Cratoxyarborenone E, gamma-Mangostin, Osthenol, a Polyketide type molecule, 4-Hydroxy-Derricin, Isobavachalcone, Methoxy-isobavachalcone, Oroxylin A, 5,7-Dimethoxy-8-prenylcoumarin, Apigenin 7-glucuronide, 3′,4′,5,7-THMethoxy3′-O-β-D-Xylopyranoside, Swietenocoumarin B, Apiin, Mulberrin, Flavaspidic acid AB, Mangostin, Phloretin, and combinations thereof. The composition may be in a form suitable for oral ingestion.

In another example, a method for inhibiting KHK-C activity in a subject may include administering a plant extract that exhibits at least IC50 (i.e., 50% KHK-C inhibition at a concentration from about 0.1 μg/mL to about 1000 μg/mL). The administering may be done to treat or prevent at least one of sugar addiction, obesity, or metabolic syndrome. The administering may be done to provide a diminished craving in the subject from at least one member selected from the group consisting of sugar, fructose, fructose-containing sugars, carbohydrates, and combinations thereof. The subject may be pre-diabetic, diabetic and/or insulin resistant.

These and other features and advantages of the invention will become apparent upon consideration of the following detailed description of the presently preferred embodiments, viewed in conjunction with the appended drawings.

### BRIEF DESCRIPTION OF THE DRAWINGS

FIG. 1 depicts an overview of the extraction process described in the application;

FIG. 2 depicts an HPLC fingerprint profile for Angelica archangelica (Wild Celery);

FIG. 3 depicts an HPLC fingerprint profile for Myrica cerifera (Bayberry);

FIG. 4 depicts an HPLC fingerprint profile for Scutellaria baicalensis (Skullcap);

FIG. 5 depicts an HPLC fingerprint profile for Petroselium crispum (Garden Parsley);

FIG. 6 depicts an HPLC fingerprint profile for Garcinia mangostana (Mangosteen);

FIG. 7 depicts an HPLC fingerprint profile for Psoralea corylifolia (Malay tea); and

FIG. 8 depicts an HPLC fingerprint profile for Morus alba (Mulberry).

### DETAILED DESCRIPTION

Throughout this disclosure, the terms “ketohexokinase” (KHK) and “fructokinase” may be used interchangeably and may refer to ketohexokinase-C (KHK-C), ketohexokinase-A (KHK-A), or combinations thereof.

As used herein, the term “administration” of a compound refers to introducing or delivering the compound to a subject to perform its intended function. The administration may be carried out by any suitable route such as orally, intranasally, parenterally (intravenously, intramuscularly, intraperitoneally, or subcutaneously), rectally, or topically.

As used herein, the term “effective amount” or “therapeutically effective amount” refers to an amount effective at dosages and for periods of time sufficient to achieve a desired result.

As used herein, the term “subject” refers to any animal (e.g., a mammal) including, but not limited to, humans, non-human primates, rodents, and the like, to which a compound may be administered.

As used herein, the term “carrier” refers to a composition that aids in maintaining one or more plant extracts in a soluble and homogeneous state in a form form suitable for administration, which is nontoxic and which does not interact with other components in a deleterious manner.

Unless indicated otherwise, all proportions and percentages recited throughout this disclosure are by weight.

A KHK-C inhibitor may be administered to a subject to inhibit KHK-C activity within the body. Such inhibition of KHK-C activity may effectively reduce the metabolization or absorption of fructose within the body. The fructose present within the body may be derived from fructose-containing sugars (e.g., fructose, sucrose, or high fructose corn syrup), sugars that can be converted to fructose within the body (e.g., sorbitol), glucose, carbohydrates (e.g., starches), or any other source. Absorption of fructose and increased KHK-C activity may contribute to a variety of conditions (e.g., obesity, metabolic syndrome, renal disease, pre-diabetes, diabetes, adenosine triphosphate (ATP) depletion, monocyte chemotactic protein-1 (MCP-1) production, insulin resistance, or intrarenal uric acid production). Inhibition of KHK-C activity may be beneficial for supporting weight management (e.g., by reducing the absorption of fructose and the associated caloric intake).

Inhibition of KHK-C activity may effectively reduce the craving for fructose from any source. A craving for fructose may result in repeated sugar intake, which may contribute to obesity, metabolic syndrome, or other conditions. Reducing the craving for fructose may be beneficial for supporting weight management (e.g., by reducing the consumption of fructose and the associated caloric intake).

In one example, a composition for inhibiting KHK-C activity may include a plant extract. The plant extract may exhibit at least IC50 (i.e., at least 50% KHK-C inhibition at a concentration from about 0.1 μg/mL to about 1000 μg/mL). In another example, the plant extract may exhibit at least 50% KHK-C inhibition at a concentration of less than about 50 μg/mL. Alternatively, the plant extract may exhibit at least 50% KHK-C inhibition at a concentration of less than about 30 μg/mL, less than about 10 μg/mL, or less than about 2 μg/mL. Preferably, the plant extract may reduce expression of a KHK-C gene or the activity of a KHK-C polypeptide by at least about 10%, preferably at least about 50%, more preferably at least about 75%, at least about 90%, or at least about 100% relative to the absence of the plant extract. The composition may be suitable for administration to a subject to support weight management. In one example, the composition may be administered to a subject to treat or prevent at least one of sugar addiction, obesity, diabetes, insulin resistance and metabolic syndrome. In one example, the composition may be administered to provide a diminished craving in the subject for at least one of sugar, fructose, fructose-containing sugars, carbohydrates, and combinations thereof. In one example, the subject may be diabetic.

The plant extract may include any suitable plant extract capable of inhibiting KHK-C activity. The plant extract may be present in the composition in an amount suitable to inhibit KHK-C activity in a subject. In one example, the plant extract may be obtained from a plant from a genus selected from the group consisting of Angelica, Cratoxylum, Myrica, Psoralea, Scutellaria, Diospyros, Andrographis, Nymphaea, Chloroxylon, Petroselinum, Morus, Pteris, Garcinia, and Malus. The plant extract may be obtained from a plant from a genus selected from the group consisting of Angelica, Cratoxylum, Myrica, Psoralea, Scutellaria, and Diospyros.

In one example, the plant extract may be obtained from a plant selected from the group consisting of Angelica archangelica, Cratoxylum prunifolium, Myrica cerifera, Psoralea corylifolia, Scutellaria baicalensis, and Diospyros attenuata, Andrographis paniculata, Nymphaea lotus, Chloroxylon swietenia, Petroselinum crispum, Morus alba, Pteris wallichiana, Garcinia mangostana, and Malus domestica. The plant extract may be obtained from a plant selected from the group consisting of Angelica archangelica, Cratoxylum prunifolium, Myrica cerifera, Psoralea corylifolia, Scutellaria baicalensis, and Diospyros attenuata.

In one example, the plant extract may include two or more plant extracts each independently obtained from a plant from a genus selected from the group consisting of, Angelica, Cratoxylum, Myrica, Psoralea, Scutellaria, Diospyros, Andrographis, Nymphaea, Chloroxylon, Petroselinum, Morus, Pteris, Garcinia, and Malus. The two or more plant extracts each independently may be obtained from a plant selected from the group consisting of, Angelica archangelica, Cratoxylum prunifolium, Myrica cerifera, Psoralea corylifolia, Scutellaria baicalensis, and Diospyros attenuata, Andrographis paniculata, Nymphaea lotus, Chloroxylon swietenia, Petroselinum crispum, Morus alba, Pteris wallichiana, Garcinia mangostana, and Malus domestica.

The composition for inhibiting KHK-C activity may include one or more compounds which may function as active ingredients. The compound may be a component of the plant extract. For example, the compound may include a phytochemical present in the plant from which the plant extract is obtained. The compound may be at least partially responsible for the inhibition of KHK-C activity exhibited by the plant extract. The compound may include any compound capable of inhibiting KHK-C activity. In one example, the compound may be selected from the group consisting of Osthol, Cratoxyarborenone E, gamma-Mangostin, Osthenol, a Polyketide type molecule, 4-Hydroxy-Derricin, Isobavachalcone, Methoxy-isobavachalcone, Oroxylin A, 5,7-Dimethoxy-8-prenylcoumarin, Apigenin 7-glucuronide, 3′,4′,5,7-THMethoxy3′-O-β-D-Xylopyranoside, Swietenocoumarin B, Apiin, Mulberrin, Flavaspidic acid AB, Mangostin, Phloretin, and combinations thereof. The compound may be selected from the group consisting of Osthol, Cratoxyarborenone E, gamma-Mangostin, Osthenol, a Polyketide type molecule, 4-Hydroxy-Derricin, Isobavachalcone, Methoxy-isobavachalcone, Oroxylin A, 5,7-Dimethoxy-8-prenylcoumarin, and combinations thereof. In one example, the compound may include a flavonoid, a polyphenol, or a combination thereof. The flavonoid may be a derivative of a phenyl-benzopyrone compound (e.g., 2-phenyl-1,4-benzopyrone, 3-phenyl-1,4-benzopyrone, or 4-phenyl-1,2-benzopyrone). In one example, the compound may include a prenylated side chain. In one example, the compound may include at least one of the functional groups I, II, or III, shown below:

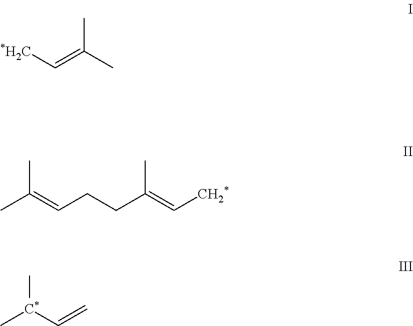

The plant extract may be commercially obtained from various sources. The plant extract may be obtained using any suitable extraction technique. Generally, any part of a plant may be used to produce the plant extract including, but not limited to, the root, the stem, the leaf, the flower, the fruit, and the fruit pod. One or more parts of the plant may be extracted to yield the plant extract. In this regard, one or more parts of the plant may be collected and milled. Thereafter, the milled material may be extracted with a suitable solvent. The solvent may be removed in a concentration step. For example, the extracted material may be screened or filtered to create a supernatant and a cake. The cake may be pressed to remove a substantial portion of the liquid, which may be added to the supernatant. The cake then may be dehydrated and used as a fiber source. The supernatant may be distilled to remove the solvent, or a portion thereof, to form a plant extract liquid concentrate. The removed solvent may be recycled. The concentrate may be dried (e.g., by spray drying) to provide a dried plant extract. The dried plant extract may be assayed and/or standardized as described herein.

The solvent may include alcohol, water, or a combination thereof. Exemplary alcoholic solvents may include, but are not limited to, C1-C7 alcohols (e.g., methanol, ethanol, propanol, isopropanol, and butanol), hydro-alcohols, or mixtures of alcohol and water (e.g., hydroethanol), polyhydric alcohols (e.g., propylene glycol and butylene glycol), and fatty alcohols. Any of these alcoholic solvents may be used in the form of a mixture. In one example, the plant extract is extracted using ethanol, water, or a combination thereof (e.g., a mixture of about 95% ethanol and about 5% water).

In one example, the plant extract may be obtained using an organic solvent extraction technique. In another example, solvent sequential fractionation may be used to obtain the plant extract. Total hydro-ethanolic extraction techniques also may be used to obtain the plant extract. Generally, this is referred to as a lump-sum extraction. The plant extract generated in the process may include a broad variety of phytochemicals present in the extracted material. The phytochemicals may be fat soluble or water soluble. Following collection of the extract solution, the solvent may be evaporated, resulting in the extract.

Total ethanol extraction also may be used. This technique uses ethanol as the solvent. This extraction technique may generate a plant extract that includes fat soluble and/or lipophilic compounds in addition to water soluble compounds.

Another example of an extraction technique that may be used to obtain the plant extract is supercritical fluid carbon dioxide extraction (SFE). In this extraction procedure, the material to be extracted may not be exposed to any organic solvents. Rather, carbon dioxide may be used as the extraction solvent, with our without a modifier, in super-critical conditions (>31.3° C. and >73.8 bar). Those of skill in the art will appreciate that temperature and pressure conditions can be varied to obtain the best yield of extract. This technique may generate an extract of fat soluble and/or lipophilic compounds, similar to a total hexane and ethyl acetate extraction technique.

The plant extract may be standardized to a specified amount of a particular compound. For example, the plant extract may be standardized to a specified amount of an active ingredient or phytochemical.

The amount of plant extract present in the KHK-C inhibiting composition may depend upon several factors, including the desired level of KHK-C inhibition, the KHK-C inhibiting level of a particular plant extract or component thereof, and other factors. Preferably, the plant extract may be present in an amount of from about 0.005 weight percent to about 50 weight percent based on the weight of the total composition.

The KHK-C inhibiting composition may include one or more acceptable carriers. The carrier may aid in enabling incorporation of the plant extract into a KHK-C inhibiting composition having a suitable form for administration to a subject. A wide number of acceptable carriers are known in the art, and the carrier may be any suitable carrier. The carrier may be suitable for administration to animals, including humans, and may be able to act as a carrier without substantially affecting the desired activity of the plant extract and/or any active ingredient. The carrier may be selected based upon the desired administration route and dosage form of the composition. For example, the composition may be suitable for use in a variety of dosage forms, such as liquid form and solid form. In one example, the composition may be provided as a gel, a syrup, a slurry, or a suspension. In one example, the composition may be provided in a liquid dosage form such as a drink shot or a liquid concentrate. In one example, the composition may be provided in a solid dosage form, such as a tablet, a pill, a capsule, a dragée, or a powder. The composition, in liquid or solid dosage form, may be in a food delivery form that is suitable for incorporation into food for delivery. Examples of suitable carriers for use in solid forms (particularly tablet and capsule forms) may include, but are not limited to, organic and inorganic inert carrier materials such as gelatin, starch, magnesium stearate, talc, gums, silicon dioxide, stearic acid, cellulose, and the like. The carrier may be substantially inert.

In one example, silicified microcrystalline cellulose may be used as a carrier. Silicified microcrystalline cellulose is a physical mixture of microcrystalline cellulose and colloidal silicon dioxide. One suitable form of silicified microcrystalline cellulose may include Prosolve 90 available from Penwest of Patterson, N.J. Silicon dioxide, in addition to that provided by the silicified microcrystalline cellulose, may be added to the composition as a processing aid. For example, silicon dioxide may be included as a glidant to improve the flow of powder during compression in the manufacturing of solid dosage units, such as tablets.

The KHK-C inhibiting composition may include other inert ingredients, such as lubricants and/or glidants. Lubricants may ease the handling of tablets during manufacturing, such as during ejection from dies. Glidants may improve powder flow during tablet compression. Stearic acid may be used as an acceptable lubricant/glidant.

The KHK-C inhibiting composition may be made in a solid dosage form, such as tablets and capsules. This form may provide a product that can be easily transported with an individual to a place of eating, such as a restaurant, and taken prior to consumption of a foodstuff. The composition may be formulated into dosage units that contain suitable amounts of the plant extract and/or active ingredient to permit an individual to determine an appropriate number of units to take based upon appropriate parameters, such as body weight, foodstuff size, or carbohydrate (e.g., sugar) content.

In one example, the KHK-C inhibiting composition may be provided in a solid dosage form (e.g., tablets or caplets) individually including from about 50 mg to about 2 g of the plant extract. The compound may be administered such that a dosage of the plant extract is from about 150 mg per day to about 2 g per day. The compound may be administered as a single dose or in multiple doses. In one example, the compound may be administered in up to three doses per day. For example, the compound may be administered prior to meals.

The dosage may be selected to provide a level of inhibitory effect in a single unit that may be effective for some individuals and/or some foodstuffs, while also allowing for relatively simple dosage increases to provide other levels of inhibitory effects that may be effective for other individuals and/or other foodstuffs.

In one example, the KHK-C inhibiting composition may be in a form adapted for oral ingestion. The form may be configured as a single dosage form intended to provide a specified dose of the plant extract. For example, the single dosage form may be a pill, a tablet, a capsule, or a drink shot. The single dosage form may include from about 50 mg to about 2 g of the plant extract.

In one example, the carrier may include saline, buffered saline, dextrose, or water. The carrier may include suitable excipients or auxiliaries to facilitate processing of the active compounds into preparations suitable for administration to a subject. The composition may be administered by any suitable route including oral, intravenous, intramuscular, intra-arterial, intramedullary, intrathecal, intraventricular, transdermal, subcutaneous, intraperitoneal, intranasal, parenteral, topical, sublingual, or rectal means. Oral dosage forms may include tablets, pills, dragées, capsules, liquids, gels, syrups, slurries, suspensions, and the like.

Certain embodiments relate to a method for inhibiting KHK-C activity in a subject comprising administering a plant extract that exhibits at least 50% KHK-C inhibition at a concentration of from about 0.1 μg/mL to about 1000 μg/mL. The administering is done to treat or prevent at least one of sugar addiction, obesity, or metabolic syndrome. The administering is also done to provide a diminished craving in the subject from at least one member selected from the group consisting of craving of sugar, fructose, fructose-containing sugars, carbohydrates, and combinations thereof. The subject may be pre-diabetic, diabetic, and or insulin resistant.

### EXAMPLES

The plant extracts identified below with reference to Table 1 are evaluated for inhibitory properties in a cell-free KHK-C model assay system. Each plant extract demonstrates meaningful inhibitory activity against KHK-C (i.e., a 50% inhibitory activity concentration in the low pM range, concentrations that are feasible within the body following oral consumption of low milligram doses). Interestingly, a number of the plant extracts possess a prenylated side chain (e.g., an isoprenyl, a geranyl, or a 1,1-dimethylallyl moiety) as part of their natural molecular backbones. The following structure illustrates one example of a compound having such prenylated side chains.

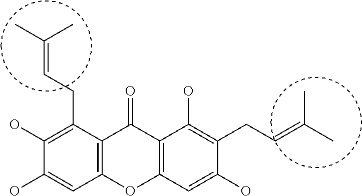 [SMILES: CC(C)=CCc1c(O)cc2oc3cc(O)c(O)c(CC=C(C)C)c3c(=O)c2c1O]

The plant extracts are screened using a 96-well high throughput enzymatic KHK assay that utilizes recombinant proteins. Recombinant proteins of human KHK-C and KHK-A are produced using the Profinity eXact fusion-tag system available from Bio-Rad Laboratories, Hercules, CA. KHK-C and KHK-A activity is assayed using a 3-step reaction. Fructose is broken down by fructokinase into fructose-1-phosphate. The ADP generated is coupled with p-enolpyruvate to generate pyruvate. The pyruvate is then coupled with NADH and broken down into NAD+ and lactate by lactate dehydrogenase. A Synergy 2 multi-mode microplate reader, available from BioTek Instruments, Inc., Winooski, Vt., is used to measure the decrease in NADH using absorbance at 340 nm (A_{340 nm}).

For the screening of the plant extracts, the KHK-C enzymatic assay is measured at 37° C. and uses 50 mM piperazine-N,N′-bis(2-ethanesulfonic acid) (PIPES), 6 mM MgCl₂, 100 mM KCI, 5 mM ATP, 2 mM phosphoenolpyruvate, 0.3 mM NADH, 10 U of pyruvate kinase, 10 U of lactate dehydrogenase, 75 ng of KHK-C, and 1 mM fructose in a total reaction volume of 200 μl. The KHK-A enzymatic assay uses the same reaction conditions except that 30 mM fructose and 50 ng/pl KHK-A are used. The mastermix (without fructose) is incubated for 5 min at 37° C. The mixture is then added to a 96-well plate containing 10 μl of the plant extracts and then incubated for 15 min at 37° C. Fructose is added to the reactions, except for the negative controls, and A_{340 nm} data is collected every minute for 1 hr. The change in absorbance during the first 30 minutes is calculated for each sample. The change in absorbance is calculated as the difference between A_{340 nm} at 0 min and A_{340 nm} at 30 min according to the following equation:

ΔA_{340 nm}=A_{340 nm}(0 min)−A_{340 nm}(30 min)

The samples are then adjusted for the negative control by calculating the difference between ΔA_{340 nm} of the respective sample and ΔA_{340 nm} of the negative control according to the following equation:

Adj ΔA_{340 nm}=ΔA_{340 nm} Sample−ΔA_{340 nm} Negative Control

The percentage inhibition of KHK-C is calculated using the following formula:

$$\frac{\begin{matrix}Adjusted Δ {A}_{340 nm} Positive Control - \\ Adjusted Δ {A}_{340 nm} Sample\end{matrix}}{Adjusted Δ {A}_{340 nm} Positive Control} \times 100$$

4-(hydroxymercuri) benzoic acid sodium salt is used as a positive inhibitory control of both KHK-C and KHK-A. Using this procedure, a 50% KHK inhibitory activity concentration (IC₅₀) is calculated for each plant extract.

Samples 1-16 shown in Table 1 are screened using the procedure described above. Each sample is an extract from the plant of the listed genus and species and exhibits the 50% KHK inhibitory activity concentration (IC₅₀) recited in Table 1.

TABLE 1

| Sample | Plate | | | | | IC₅₀ |
|---|---|---|---|---|---|---|
| No. | ID | Well ID | Genus | Species | Description | (μM) |
| 1 | UCO- | G10 | Angelica | archangelica | European | 0.53 |
| | 02 | | | | medicinal | |
| | | | | | plant | |
| 2 | UCO- | C08 | Cratoxylum | prunifolium | tree from | 1.40 |
| | 02 | | | | SE Asia | |
| 3 | UCO- | F02 | Cratoxylum | prunifolium | tree from | 1.97 |
| | 04 | | | | SE Asia | |
| 4 | UCO- | G10 | Angelica | archangelica | European | 5.97 |
| | 05 | | | | medicinal | |
| | | | | | plant | |
| 5 | UCO- | B08 | Myrica | cerifera | medicinal | 6.20 |
| | 02 | | | | plant from | |
| | | | | | SE N | |
| | | | | | America | |
| 6 | UCO- | B11 | Psoralea | corylifolia | Ayurvedic | 6.20 |
| | 10 | | | | medicinal | |
| | | | | | plant | |
| 7 | UCO- | E01 | Scutellaria | baicalensis | Chinese | 7.70 |
| | 03 | | | | medicinal | |
| | | | | | plant | |
| 8 | UCO- | G07 | Diospyros | attenuata | tree from E | 9.40 |
| | 01 | | | | Africa | |
| 9 | UCO- | F03 | Andrographis | paniculata | Chinese | 24.10 |
| | 04 | | | | medicinal | |
| | | | | | plant | |
| 10 | UCO- | D10 | Nymphaea | lotus | plant from | 26.00 |
| | 01 | | | | SE Asia | |
| | | | | | and E | |
| | | | | | Africa | |
| 11 | UCO- | G6 | Chloroxylon | swietenia | threatened | 26.00 |
| | 13 | | | | species | |
| 12 | UCO-06 | E01 | Petroselinum | crispum | edible | 40.60 |
| 13 | UCO-03 | D07 | Morus | alba | edible | 44.20 |
| 14 | UCO- | F08 | Pteris | wallichiana | fern from | 44.90 |
| | 02 | | | | SE Asia | |
| 15 | FTL | B09 | Garcinia | mangostana | tree from | 30 ug/mL |
| | plate 8 | | | | SE Asia | |
| 16 | FTL | E10 | Malus | domestica | apple tree | 9 ug/mL |
| | plate 9 | | | | | |

Table 2 shows a phytochemical present in each of samples 1-17, including the structure of each phytochemical.

TABLE 2

| Sample | | | |
|---|---|---|---|
| No. | Phytochemical | CAS | Structure |
| 1 | Osthol | 484- 12-8 | 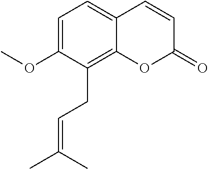 [SMILES: COc1ccc2ccc(=O)oc2c1CC=C(C)C] |
| 2 | Cratoxyarborenone E | | 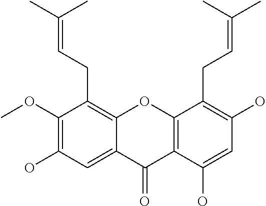 [SMILES: COc1c(O)cc2c(=O)c3c(O)cc(O)c(CC=C(C)C)c3oc2c1CC=C(C)C] |
| 3 | gamma-Mangostin | 31271- 07-5 | 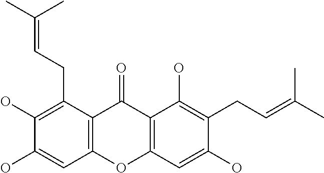 [SMILES: CC(C)=CCc1c(O)cc2oc3cc(O)c(O)c(CC=C(C)C)c3c(=O)c2c1O] |
| 4 | Osthenol | 484- 14-0 | 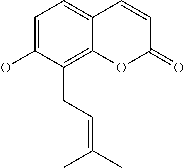 [SMILES: CC(C)=CCc1c(O)ccc2ccc(=O)oc12] |
| 5 | Polyketide type molecule | | 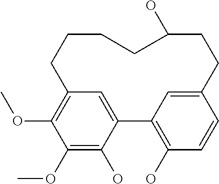 [SMILES: COc1c2cc(c(O)c1OC)-c1cc(ccc1O)CCC(O)CCCC2] |
| 6 | 4-Hydroxy- Derricin, Isobavachalcone | 20784- 50-3 | 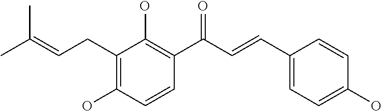 [SMILES: CC(C)=CCc1c(O)ccc(C(=O)/C=C/c2ccc(O)cc2)c1O] |
| 7 | Methoxy- Isobavachalcone | | 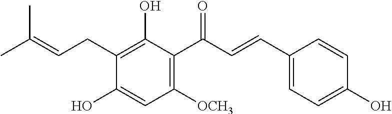 [SMILES: COc1cc(O)c(CC=C(C)C)c(O)c1C(=O)/C=C/c1ccc(O)cc1] |
| 8 | Oroxylin A | 480- 11-5 | 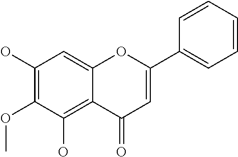 [SMILES: COc1c(O)cc2oc(-c3ccccc3)cc(=O)c2c1O] |
| 9 | 5,7-Dimethoxy-8- prenylcoumarin | 17245- 25-9 | 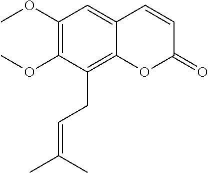 [SMILES: COc1cc2ccc(=O)oc2c(CC=C(C)C)c1OC] |
| 10 | Apigenin 7- glucuronide | 29741- 09-1 | 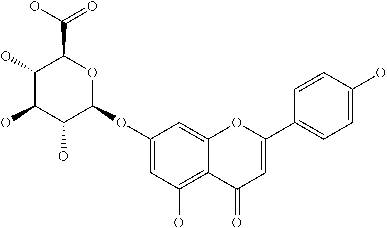 [SMILES: O=C(O)[C@H]1O[C@@H](Oc2cc(O)c3c(=O)cc(-c4ccc(O)cc4)oc3c2)[C@H](O)[C@@H](O)[C@@H]1O] |
| 11 | 3′,4′,5,7- THMethoxy3′-O-β- D-Xylopyranoside | 93373- 16-1 | 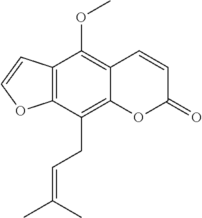 [SMILES: COc1c2ccoc2c(CC=C(C)C)c2oc(=O)ccc12] |
| 12 | Swietenocoumarin B | 64652- 23-9 | 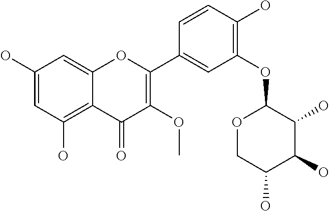 [SMILES: COc1c(-c2ccc(O)c(O[C@@H]3OC[C@@H](O)[C@H](O)[C@H]3O)c2)oc2cc(O)cc(O)c2c1=O] |
| 13 | Apiin | 26544- 34-3 | 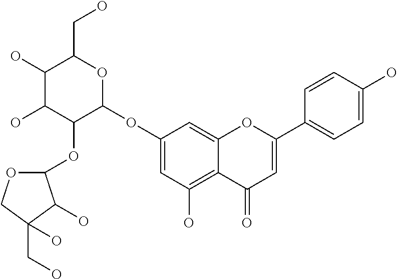 [SMILES: O=c1cc(-c2ccc(O)cc2)oc2cc(OC3OC(CO)C(O)C(O)C3OC3OCC(O)(CO)C3O)cc(O)c12] |
| 14 | Mulberrin | 62949- 79-5 | 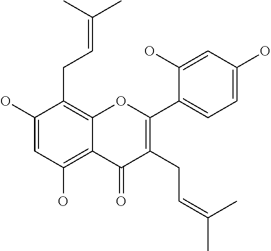 [SMILES: CC(C)=CCc1c(-c2ccc(O)cc2O)oc2c(CC=C(C)C)c(O)cc(O)c2c1=O] |
| 15 | Flavaspidic acid AB | 3761- 64-6 | 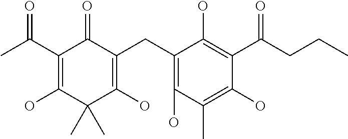 [SMILES: CCCC(=O)c1c(O)c(C)c(O)c(CC2=C(O)C(C)(C)C(O)=C(C(C)=O)C2=O)c1O] |
| 16 | Mangostin | | 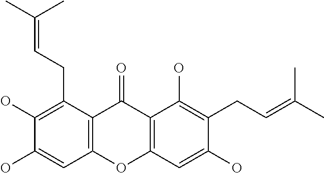 [SMILES: CC(C)=CCc1c(O)cc2oc3cc(O)c(O)c(CC=C(C)C)c3c(=O)c2c1O] |
| 17 | Phloretin | | 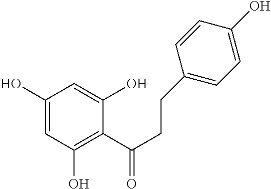 [SMILES: O=C(CCc1ccc(O)cc1)c1c(O)cc(O)cc1O] |

### EXAMPLES

### Example 1

### Method of Extraction: Preparation of Three Fractions, Hydrophilic, Lipophilic and Mixed/Combination Fractions for Biological in vitro High through-put Screening.

Reagents/Solutions

General Chemistry Laboratory supply and standard equipment.

Deionized water (DI). HPLC grade or equivalent.

Chloroform, (Trichloromethane), ACS grade. Fisher Scientific # C298-4 or equivalent.

Methanol, Optima grade, Fisher Scientific # A456-4 or equivalent.

Plant Materials: All plant materials used in this study were obtained from Applicants' farms in the form of dry powders.

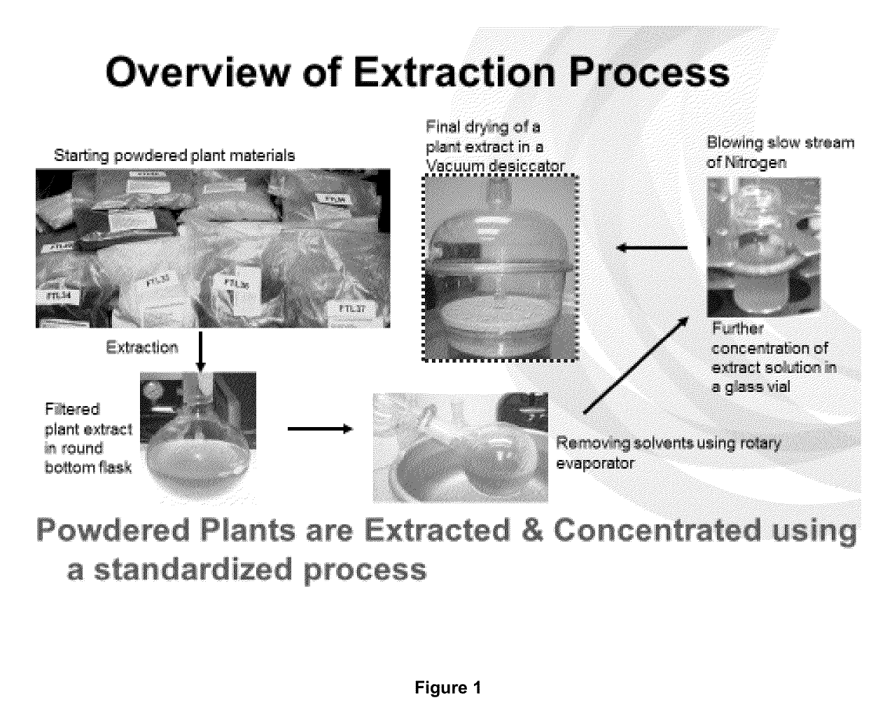

FIG. 1 shows a general schematic diagram of the extraction procedures described below.

A. Preparation of Hydrophilic Fractions:

Approximately 50 g, to the nearest 0.01 g, of the powdered botanical was weighed into a wide mouth 500 mL Erlenmeyer flask. A stir bar was added and 300 mL of methanol was poured into the flask. The flask was loosely covered using aluminum foil. The flask was placed on a magnetic stir plate and stirred for 12 hours (minimum) using a slow/medium stir rate. The samples were kept out of direct light. Next, the flask was removed from the stir plate and sonicated for one hour, with occasional swirling, at room temperature. The sample solution was then filtered through GF/A Filter Paper directly into a 500 mL round flat bottom boiling flask. The filter paper was scraped and botanical residue was collected from the filter paper onto an aluminum weigh boat (or foil). The sample was dried at room temperature in a hood for at least 12 hours and the residue stored in an appropriate container. Next, using a graduated cylinder, 100 mL aliquot of this sample solution was pipetted out in an Erlenmeyer flask and capped with aluminum foil and stored in a refrigerator. The sample was then properly identified. This solution was then used for the preparation of a combination fraction in “Section C” below of this procedure.

Using a rotary evaporator, the remaining solvent was evaporated in the round bottom boiling flask. The volume of the solvent was reduced to less than 10 mL. The concentrated extract (still in liquid form) was then transferred, using a glass pipette, to a pre-weighed scintillation vial (weigh without cap). Methanol was used for further dilution for transfer purposes as needed.

The vial was then placed under nitrogen evaporator to reduce volume to as minimum as possible (using slow stream of nitrogen). Recommended water bath temperature should be around 40° C. The vial was then removed from the nitrogen evaporator and placed in a vacuum desiccator until dry (approximately 12 hours). The final dry weight (without cap) (check for constant weight) of the fraction was recorded in the scintillation vial and the weight of the fraction (by difference) was also recorded.

B. Preparation of Lipophilic Fractions:

Approximately 50 g, to the nearest 0.01 g, of the powdered botanical was weighed into a wide mouth 500 mL Erlenmeyer flask. A stir bar was added and 300 mL of chloroform was poured into the flask. The flask opening was covered using aluminum foil. The flask was then placed on a magnetic stir plate and stirred for 12 hours (minimum) using a slow/medium stir rate. The samples were kept out of direct light. The flask was then removed from the stir plate and the sample was sonicated for one hour, with occasional swirling, at room temperature. Next, the sample solution was filtered through GF/A Filter Paper directly into a 500 mL round, flat bottom boiling flask. Using a graduated cylinder, a 100 mL aliquot of this solution was removed and stored in a foiled Erlenmeyer flask in a refrigerator for further step. This solution was then used for the preparation of a combination fraction in “Section C” of this procedure.

Using a rotary evaporator, the remaining solvent in the round bottom boiling flask was evaporated. The volume of the solvent was reduced to less than 10 mL. The concentrated extract, (still in a liquid form) was then transferred using a glass pipette, to a pre-weighed scintillation vial (weigh vial without cap). Chloroform was used for further dilution and transfer purposes as needed.

Next, to prepare a lipophilic fraction, the vial was placed under a nitrogen evaporator to reduce volume to as minimum as possible (using slow stream of nitrogen). Recommended water bath temperature should be around 40° C. The vial was then removed from the nitrogen evaporator and placed in a vacuum desiccator until dry (approximately 12 hours). The final dry weight (without cap) (check for constant weight) of the fraction was recorded in the scintillation vial and the weight of the fraction (by difference) was also recorded.

C. Preparation of a Mixed/Combination Fractions:

The two 100 mL aliquots of hydrophilic and lipophilic solutions saved during Preparations A and B above were combined. Specifically, a 100 mL aliquot of the sample solution from “Section A” above and 100 mL aliquot of the solution from “Section B” were combined into a 500 mL round bottom boiling flask and mixed well. Using a rotary evaporator, the solvent in the sample concentrate was evaporated. The volume of the solvent was reduced to less than 10 mL. The concentrated extract (still in liquid form) was transferred using a glass pipette to a pre-weighed scintillation vial (weigh without cap). A mixture of chloroform/methanol (1/1 v/v) was used for further dilution for transfer purposes as needed.

Next, the vial was placed under a nitrogen evaporator and the volume was reduced to minimum as possible (using slow stream of nitrogen). Recommended water bath temperature should be around 40° C. The vial was then removed from the nitrogen evaporator and placed in a desiccator until dry (approximately 12 hours) and cooled to room temperature. The final dry weight (without cap) (check for constant weight) of the fraction in the scintillation vial (by difference) was recorded.

The sample was then properly identified.

The vial was stored in a refrigerator for future use.

D. HPLC Methodology:

Materials and Instrumentation:

All solvents were HPLC grade and purchased from Fisher Scientific. HPLC separation was achieved using an Agilent Technologies, Santa Clara, Calif. HP1100 System equipped with Photodiode-array detection and Chemstation software using Waters C 18 4um NovaPak column (250×4.6 mm) Part No 0528401. Botanical samples were fingerprinted with 0.2% ortho-phosphoric acid (OPA) v/v with deionized (DI) water and acetonitrile (ACN) elution gradient as outlined in the Table 1.

TABLE 1
(HPLC Conditions):
HPLC Gradient:

| | 0.2% o-Phosphoric acid | ACN |
|---|---|---|
| Time | (OPA) in DI water | (Acetonitrile) |
| 0.0 | 92 | 8 |
| 12 | 90 | 10 |
| 14 | 88 | 12 |
| 26 | 76 | 24 |
| 34 | 60 | 40 |
| 37 | 60 | 40 |
| 40 | 92 | 8 |
| 42 | 92 | 8 |

| | |
|---|---|
| Flow Rate: | 1.0 mL/min |
| Column Temperature: | Ambient |
| Injection Volume: | 10 μL |
| Detection Wavelengths: | 210-370 nm |
| Integration: | Peak area |
| Run Time: | 42 minutes |

Sample Preparation:

Approximately 300 mg of dry powdered sample of botanical extracts was weighted, to the nearest 0.1 mg, into a 50 mL volumetric flask. About 40 mL of 80/20 Methanol in DI Water (diluent) was added and the mixture was shaken well to dissolve. The flask was then placed in a sonic bath and sonicated for 10 minutes. The mixture was then cooled to room temperature, diluted to volume with diluent and mixed thoroughly. The sample solution was then filtered with a disposable syringe through a 0.45 micron PVDF filter into an HPLC auto sampler vial.

Results:

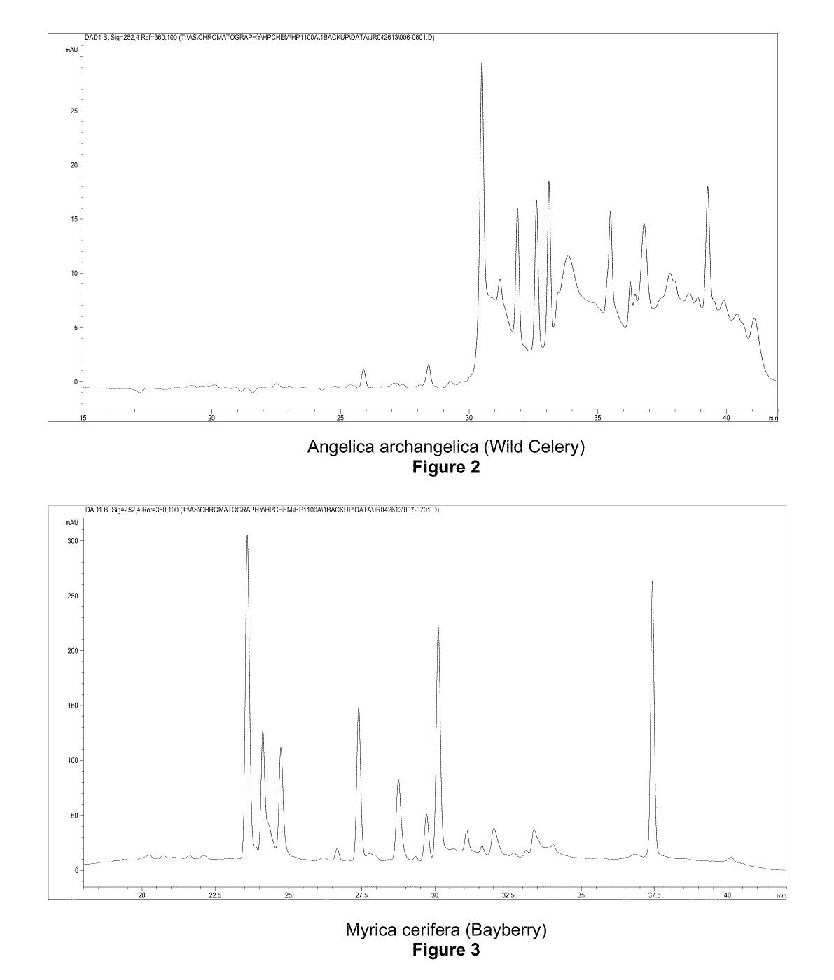

Typical HPLC fingerprint profiles of the tested botanicals are shown in FIGS. 2-8.

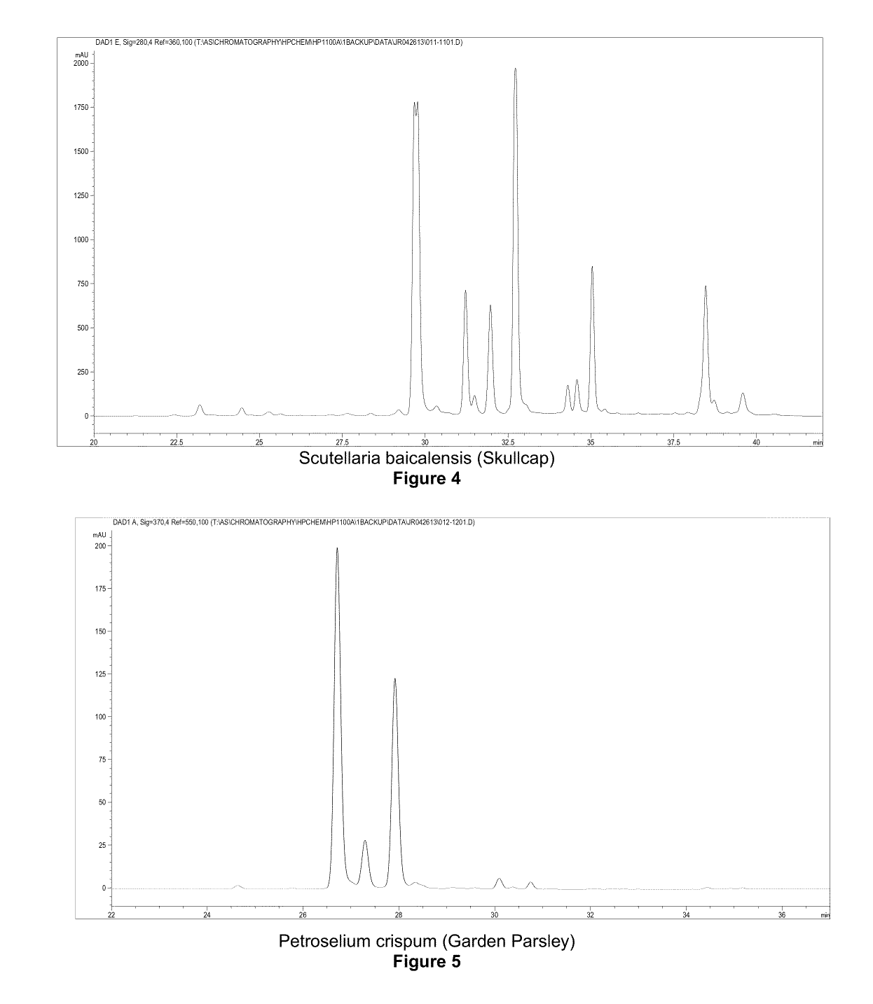

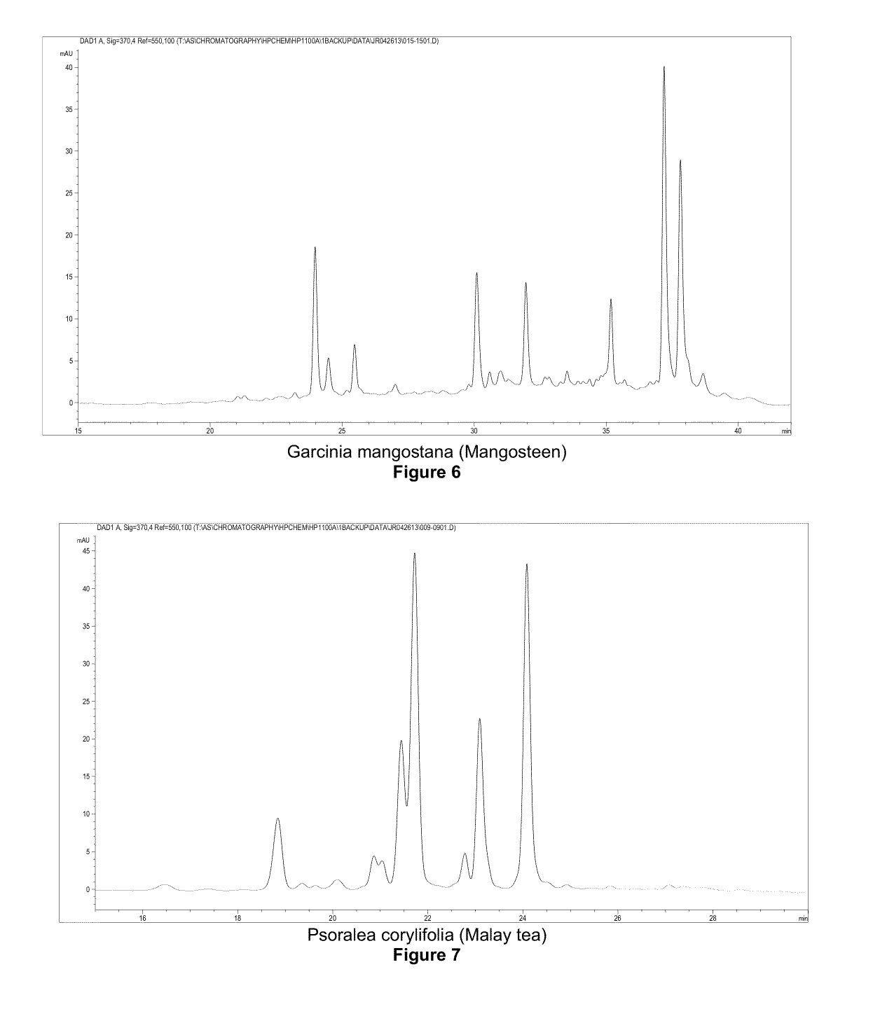

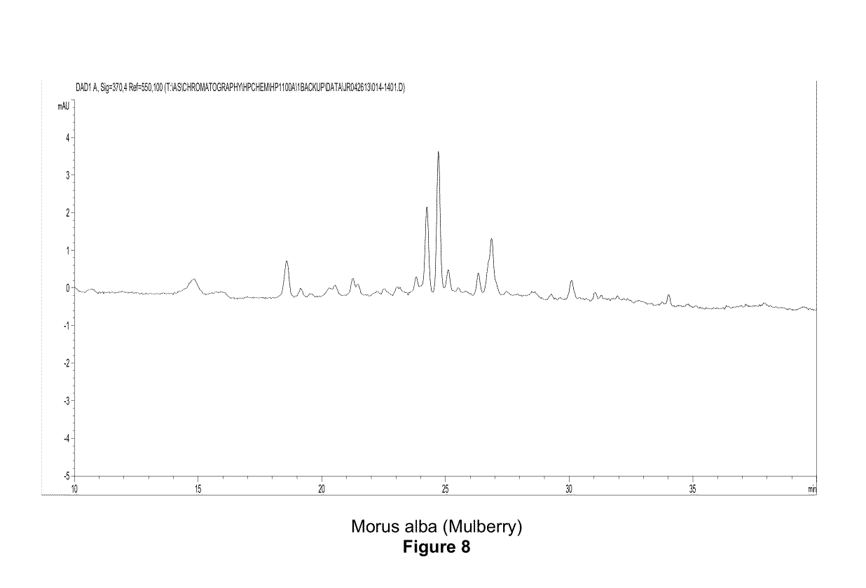

Specifically, an HPLC fingerprint profile for Angelica archangelica (Wild Celery) is shown in FIG. 2; an HPLC fingerprint profile for Myrica cerifera (Bayberry) is shown in FIG. 3; an HPLC fingerprint profile for Scutellaria baicalensis (Skullcap) is shown in FIG. 4; an HPLC fingerprint profile for Petroselium crispum (Garden Parsley) is shown in FIG. 5; an HPLC fingerprint profile for Garcinia mangostana (Mangosteen) is shown in FIG. 6; an HPLC fingerprint profile for Psoralea corylifolia (Malay tea) is shown in FIG. 7; and an HPLC fingerprint profile for Morus alba (Mulberry) is shown in FIG. 8.

While the present invention has been described with reference to specific exemplary embodiments, it will be evident that various modifications and changes may be made to these embodiments without departing from the spirit and scope of the invention. It is the following claims, including all equivalents, which are intended to define the spirit and scope of the invention.

We claim:

1. A composition for inhibiting ketohexokinase-C (KHK-C) activity comprising a plant extract exhibiting at least IC50, wherein at least 50% KHK-C inhibition occurs at a concentration from about 0.1 μg/mL to about 1000 μg/mL.

2. The composition of claim 1, wherein the plant extract is obtained from a plant from a genus selected from the group consisting of Angelica, Cratoxylum, Myrica, Psoralea, Scutellaria, Diospyros, Andrographis, Nymphaea, Chloroxylon, Petroselinum, Morus, Pteris, Garcinia, and Malus.

3. The composition of claim 2, wherein the plant extract is obtained from a plant selected from the group consisting of Angelica archangelica, Cratoxylum prunifolium, Myrica cerifera, Psoralea corylifolia, Scutellaria baicalensis, and Diospyros attenuata, Andrographis paniculata, Nymphaea lotus, Chloroxylon swietenia, Petroselinum crispum, Morus alba, Pteris wallichiana, Garcinia mangostana, and Malus domestica.

4. The composition of claim 1, wherein the plant extract is obtained from a plant from a genus selected from the group consisting of Angelica, Cratoxylum, Myrica, Psoralea, Scutellaria, and Diospyros.

5. The composition of claim 4, wherein the plant extract is obtained from a plant selected from the group consisting of Angelica archangelica, Cratoxylum prunifolium, Myrica cerifera, Psoralea corylifolia, Scutellaria baicalensis, and Diospyros attenuata.

6. The composition of claim 1, wherein the plant extract comprises a compound selected from the group consisting of Osthol, Cratoxyarborenone E, gamma-Mangostin, Osthenol, a Polyketide type molecule, 4-Hydroxy-Derricin, Isobavachalcone, Methoxy isobavachalcone, Oroxylin A, 5,7-Dimethoxy-8-prenylcoumarin, Apigenin 7-glucuronide, 3′,4′,5,7-THMethoxy3′-O-β-D-Xylopyranoside, Swietenocoumarin B, Apiin, Mulberrin, Flavaspidic acid AB, Mangostin, Phloretin, and combinations thereof.

7. The composition of claim 1, wherein the plant extract comprises a compound having a prenylated side chain.

8. The composition of claim 1, wherein an amount of the plant extract in the composition is between about 0.005 weight percent and about 50 weight percent.

9. The composition of claim 1, wherein the composition is in a form suitable for oral ingestion.

10. The composition of claim 9, wherein the form is selected from the group consisting of a pill, a tablet, a capsule, a caplet, a dragée, a powder, a liquid, a gel, a syrup, a slurry, and a suspension.

11. The composition of claim 1, wherein the plant extract comprises at least one of the following functional groups I, II, or III:

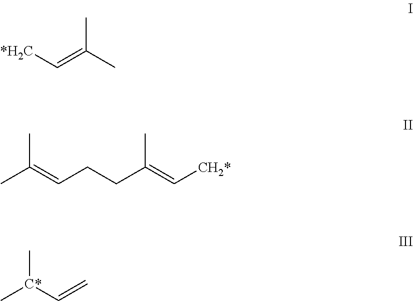

12. A method for inhibiting KHK-C activity in a subject comprising administering a plant extract that exhibits at least 50% KHK-C inhibition at a concentration of from about 0.1 μg/mL to about 1000 μg/mL.

13. The method of claim 12, wherein the administering is done to treat or prevent at least one of sugar addiction, obesity, or metabolic syndrome.

14. The method of claim 12, wherein the administering is done to provide a diminished craving in the subject from at least one member selected from the group consisting of sugar, fructose, fructose-containing sugars, carbohydrates, and combinations thereof.

15. The method of claim 12 wherein the subject is pre-diabetic.

16. The method of claim 12, wherein the subject is diabetic.

17. The method of claim 12, wherein the subject is insulin resistant.
